(19) United States
(12) Patent Application Publication
(10) Pub. No.: US 2016/0193348 A1
(43) Pub. Date: Jul. 7, 2016

(54) MUCOUS LAYER-ADHESIVE POLY-r-GLUTAMIC ACID NANOMICELLES AND DRUG DELIVERY SYSTEM USING SAME

(71) Applicants: BIOLEADERS CORPORATION, Daejeon (KR); THE INDUSTRY AND ACADEMIC COOPERATION IN CHUNGNAM NATIONAL UNIVERSITY, Deajeon (KR); KOREA RESEARCH INSTITUTE OF BIOSCIENCE AND BIOTECHNOLOGY, Daejeon (KR); KOOKMIN UNIVERSITY INDUSTRY-ACADEMIC COOPERATION FOUNDATION, Seoul (KR)

(72) Inventors: Jae-Chul Choi, Daejeon (KR); Yong Taik Lim, Daejeon (KR); Haryoung Poo, Daejeon (KR); Moon-Hee Sung, Seoul (KR)

(21) Appl. No.: 14/894,413

(22) PCT Filed: Jun. 4, 2013
(86) PCT No.: PCT/KR2013/004927

§ 371 Date: Feb. 2, 2016

(30) Foreign Application Priority Data
May 31, 2013 (KR) 10-2013-0062734

(51) Int. Cl. A61K 47/48; C12N 7/00; A61K 39/385; A61K 39/145; A61K 33/18; A61K 39/00

(52) U.S. Cl. CPC A61K 47/488; A61K 33/18; A61K 39/0005; A61K 39/385; A61K 39/145; C12N 7/00; A61K 47/4833; A61K 47/48238; A61K 47/48123; C12N 2760/16134

(57) ABSTRACT

The present invention relates to nanomicelles composed of a complex of a lipophilic compound and a poly-gamma-glutamic acid wherein a portion of carboxyl groups are substituted with an amine group, and more particularly, to nanomicelles composed of a complex of a lipophilic compound and a poly-gamma-glutamic acid wherein a portion of carboxyl groups are substituted with an amine group, a preparation method thereof, and a drug delivery system employing the mucous membrane-adhesive property of the nanomicelles. According to the present invention, the nanomicelle drug delivery system based on poly-gamma-glutamic acid that is a natural biopolymer can be used for the delivery of a drug to mucous membranes to thereby increase the in vivo stability and effectiveness of the drug.

### TECHNICAL FIELD

The present invention relates to nanomicelles composed of a complex of a lipophilic compound and a poly-gamma-glutamic acid wherein a portion of carboxyl groups are substituted with an amine group, and more particularly, to nanomicelles composed of a complex of a lipophilic compound and a poly-gamma-glutamic acid wherein a portion of carboxyl groups are substituted with an amine group, a preparation method thereof, and a drug delivery system employing the mucous membrane-adhesive property of the nanomicelles.

### BACKGROUND ART

The present invention relates to poly-gamma-glutamic nanomicelles capable of delivering drugs or fluorophores using poly-gamma-glutamic acid that is a biocompatible natural polymer, and a preparation method thereof. Among drug administration routes, an oral administration route is most frequently used. However, in the case of drugs whose therapeutic effects increase when administered directly to mucous membranes and drugs whose bioavailability effectively increases when absorbed through mucous membranes, mucous membranes are considered as a major alternative administration route. Mucous membranes are found in the mouse, the nasal cavity, the reproductive organs, the rectum, digestive organs, and skin ulcer sites, as well as sites that are not exposed to the outside of the body, including gastrointestinal tracts. These physiologically active substances that are administered to mucous membranes have an advantage over general drugs for oral administration in that they can be absorbed directly into the blood flow, and thus have a short onset time of pharmaceutical effects. In addition, in the case of oral mucous membranes, nasal mucous membranes, respiratory mucous membranes, eye mucous membranes, reproductive mucous membranes, and skin ulcer sites, the direct application of drugs to lesions of the mucous membranes has an advantage over oral administration in that it can further increase the effectiveness of the drugs.

In addition, because many immune cells are concentrated around mucous membranes, it is very important to enhance mucosal immunity by delivering influenza vaccine antigens through mucous membranes. In this regard, if materials that are used as antigen delivery systems can function as vaccine adjuvants that assist in activation of immune cells, in addition to functioning to increase the delivery of antigens, they will have greater values.

As mucous membrane-adhesive polymers, materials based on polyacrylic acid have been frequently used. U.S. Pat. No. 4,292,299 discloses a mucous membrane-adhesive drug delivery system comprising a copolymer of polyacrylic acid or its derivative and a polyacrylic acid cellulose derivative. European Patent No. 0 654 261 discloses the use of a physical mixture of a cellulose ether derivative, a polyacrylic acid derivative and gelatin to obtain the property of adhering to mucous membranes. In addition, U.S. Pat. No. 5,942,243 discloses the use of a polystyrene-based copolymer in preparations for mucosal administration. However, these mucous membrane-adhesive materials are mostly organic synthetic materials prepared by chemical synthesis methods. Thus, the development of mucous membrane-adhesive delivery systems based on biocompatible biomaterials has been actively made.

Mucin, which is the major component of mucus, collectively refers to viscous substances secreted from animal exocrine glands, and is a complex glycoprotein. It is known that mucin exhibits useful effects in vivo by promoting the digestion of cellular proteins, and has the effects of protecting the stomach wall and neutralizing poison. Organs in which mucin is present include the oral cavity, the nasal cavity, the larynx, gastrointestinal tracts, eyes, the anus, and the vagina, and the thickness thereof ranges from several nanometers to 170 micrometers. The structure of the mucin network is maintained by various bonds, including ionic bonds, hydrogen bonds, disulfide bonds, van der Waals bonds, and entanglement between mucin molecules. Studies on mucus adhesive polymers which are connected with such bonds have been actively conducted, and polymers having the property of strongly interacting with bonds present in the mucin layer have been mainly studied. The mucus layer generally has a complex porous network structure. It is known that and bacteria having a size of several micrometers generally cannot pass through the mucus layer, but penetrates a portion of the mucus layer, which was destroyed or is thin in thickness. Although antibodies having a size of about 10 nm and plasmid DNAs larger than the antibodies are able to pass through the mucus layer, these are difficult to pass through the mucus layer due to the degradation by many enzymes present in the mucus layer. It is known that viruses having a size of 200 nm or less can quickly pass through the mucus layer.

Poly-gamma-glutamic acid that is used in the present invention is a polypeptide having a carboxyl group, and is produced using a salt-tolerant Bacillus subtilis Chungkookjang strain that produces high-molecular-weight poly-gamma-glutamic acid (Korean Patent No. 500796). In addition, patent applications relating to anticancer compositions, immune adjuvants, immune enhancers, and virus infection inhibitors, which contain poly-gamma-glutamic acid, have been filed (Korean Patent Nos. 496606, 517114, 475406, and 873179). Further, as studies on the medicinal use of substances have been continuously conducted, the various effects of substances have been continuously found. Accordingly, the present inventors have made extensive efforts to develop a drug delivery system having the property of adhering to mucous membranes, and as a result, have found that nanomicelles composed of a complex of a lipophilic compound and a poly-gamma-glutamic acid wherein a portion of carboxyl groups are substituted with an amine group has the property of adhering to mucous membranes and the effect of enhancing immunity, and also found that, when the nanomicelles are used as a drug delivery system, the delivery of drugs to mucous membranes is improved, thereby completing the present invention.

### DISCLOSURE OF INVENTION

### Technical Problem

It is an object of the present invention to provide nanomicelles, which are based on the natural biopolymer poly-gamma-glutamic acid and have an excellent property of adhering to mucous membranes, and a preparation method thereof.

Another object of the present invention is to provide a drug delivery system comprising nanomicelles having an excellent property of adhering to mucous membranes.

### Technical Solution

To achieve the above objects, the present invention provides nanomicelles composed of a complex of a lipophilic compound and a poly-gamma-glutamic acid wherein a portion of carboxyl groups are substituted with an amine group.

The present invention also provides a method for preparing nanomicelles composed of a complex of a lipophilic compound and a poly-gamma-glutamic acid wherein a portion of carboxyl groups are substituted with an amine group, the method comprising the steps of:

(a) mixing a poly-gamma-glutamic acid solution with a lipophilic compound-amine complex solution to prepare a poly-gamma-glutamic acid-lipophilic compound complex; and

(b) treating the poly-gamma-glutamic acid-lipophilic compound complex with an amine-based compound to substitute the carboxyl group of the poly-gamma-glutamic acid with an amine group, thereby preparing nanomicelles composed of a complex of a lipophilic compound and a poly-gamma-glutamic acid wherein a portion of carboxyl groups are substituted with an amine group.

The present invention also provides a nanomicelle drug delivery system wherein a drug selected from the group consisting of proteins, genes, peptides, compounds, antigens and natural materials is loaded in nanomicelles composed of a complex of a lipophilic compound and a poly-gamma-glutamic acid wherein a portion of carboxyl groups are substituted with an amine group.

### BRIEF DESCRIPTION OF THE DRAWINGS

FIG. 1a shows the chemical formula of mucous membrane-adhesive poly-gamma-glutamic acid (γ-PGA)-cholesterol nanomicelles according to an embodiment of the present invention, and is a conceptual view showing a principle by which the nanomicelles deliver a virus antigen; FIG. 1(b) shows the results of dynamic light scattering (DLS) analysis performed to measure the particle diameter of the nanomicelles; and FIG. 1(c) shows the results of cryo-TEM analysis of the nanomicelles.

FIG. 2 shows SPECT/CT images (FIG. 2(a)) and immunohistofluorescence images (FIG. 2(b)) demonstrating the mucous membrane adhesive property of poly-gamma-glutamic acid-cholesterol nanomicelles injected intranasally to a mouse model.

FIG. 3 shows the results of measuring antigen-specific immunity in mouse models after intranasal injection of poly-gamma-glutamic acid-cholesterol nanomicelles loaded with a model antigen (OVA). Specifically, FIG. 3(a) shows the results of measuring the ability to induce OVA-specific IgG antibody responses. For comparison, FIG. 3(a) shows antigen-specific immune responses that appear in the case of a poly-gamma-glutamic acid (γ-PGA)-cholesterol complex (whose surface is composed of carboxyl groups) wherein poly-gamma-glutamic acid is substituted only with a lipophilic group such as cholesterol. FIG. 3(b) shows the results of measuring the ability to induce OVA-specific IgA antibody responses, and FIG. 3(c) shows the results of measuring the number of IFN-γ-producing cells by an IFN-γ ELISpot assay.

FIG. 4 shows the results of measuring antigen-specific immune immunity after intranasal injection of poly-gamma-glutamic acid-cholesterol nanomicelles loaded with an influenza virus antigen (1. PBS, 2. PR8 only, 3. PR8 plus γ-PGA nanomicelles (10 μg), and 4. PR8 plus γ-PGA nanomicelles (100 μg). Specifically, FIG. 4(a) shows the results of measuring the production of influenza virus-specific IgG antibody; FIG. 4(b) shows the results of measuring the production of influenza virus-specific IgA antibody; FIG. 4(c) shows the results of measuring the number of IFN-γ-producing cells by an IFN-γ ELISpot assay; FIG. 4(d) shows the results of measuring the effect of a virus antibody on inhibition of hemagglutination; FIG. 4(e) shows the change in the mouse body weight caused by viral infection after vaccine administration; and FIG. 4(f) shows the results of measuring the lethality of mice caused by viral infection after vaccine administration (*, p<0.05; **, p<0.01; ***, p<0.001; n. s., not significant).

### BEST MODE FOR CARRYING OUT THE INVENTION

In one aspect, the present invention is directed to nanomicelles composed of a complex of a lipophilic compound and a poly-gamma-glutamic acid wherein a portion of carboxyl groups are substituted with an amine group, and to a preparation method thereof.

According to the present invention, the nanomicelles composed of a complex of a lipophilic compound and a poly-gamma-glutamic acid wherein a portion of carboxyl groups are substituted with an amine group is prepared by the following steps:

(a) mixing a poly-glutamic acid solution with a lipophilic compound-amine complex solution to prepare a poly-gamma-glutamic acid-lipophilic compound complex; and

(b) treating the poly-glutamic acid-lipophilic compound complex with an amine-based compound to substitute the carboxyl group of the poly-gamma-glutamic acid with an amine group, thereby preparing nanomicelles composed of a complex of a lipophilic compound and a poly-gamma-glutamic acid wherein a portion of carboxyl groups are substituted with an amine group.

The lipophilic compound that is used in the present invention may be selected from the group consisting of cholesterols and their derivatives, aliphatic compounds having 3 to 21 carbon atoms (C₃-C₂₁), and aromatic compounds containing 1 to 10 benzene groups.

For example, among lipophilic compounds, cholesterol-based compounds may be selected from cholesterols and their derivatives, including cholesterol, cholesterol β-D-glucoside, cholesteryl N-(trimethylammonioethyl)carbamate chloride, cholesteryl oleyl carbonate, Cholestanol, Lanosterol, Clofibrate, Cholesteryl oleate, Mevastatin, Thiocholesterol, Stigmastanol, Cholesteryl palmitate, Diethylumbelliferyl phosphate, Cholesteryl heptadecanoate, β-Sitosterol, 5α-Cholestane, and 5-Cholesten-3-one.

Among lipophilic compounds, tocopherol-based compounds may be selected from C₁₀-C₁₀₀ tocopherols and their precursors and derivatives, including (±)-α-tocopherol, (+)-δ-tocopherol, DL-α-tocopherol acetate, (+)-γ-Tocopherol, Vitamin E acetate, Pinobanksin, Methyl jasmonate, and Retinyl acetate.

Among lipophilic compounds, aliphatic compounds may be selected from C₃ hydrophobic compounds and their derivatives, including propyne, acrolein, allyl alcohol, 2-propene-1-thiol, propane, isopropylamine, 1,3-diaminopropane, N-isopropylmethylamine, propylene glycol, 2-nitropropane, and 1-nitropropane; C₄ hydrophobic compounds and their derivatives, including butyne, butyraldehyde, 2-methyl-1-propanol, diethyl ether, 2-butanol, 1-butanol, and butyl alcohol; and C₅ hydrophobic compounds and their derivatives, including pentyl acetate, 2-pentyl acetate, 2-pentyl-2-cyclopenten-1-one, pentyl nitrite, pentyl propionate, pentyl valerate, N-(1-pentyl) formamide, pentyl laurate, and 2-pentyl butyrate.

Among lipophilic compounds, aromatic compounds containing one or more benzene group may be selected from aliphatic and non-aromatic cyclic compounds based on benzene, cyclohexane, hexane or the like, aromatic compounds and their derivatives, and cyclic and non-cyclic structures, including benzyl chloride, benzyl chloroformate, benzyl formate, benzyl methyl ether, benzyl propionate, phenol, 2-(methylamino)phenol, 2-tert-butyl-6-methyl-phenol, 3-(trifluoromethyl)phenol, dansylcadaverine, 3-(dansylamino)phenylboronic acid, hexane, 3-oxabicyclo[3.1.0]hexane-2,4-dione, 1,6-bis(trichlorosilyl)hexane, cyclopentene oxide, 4-hexylphenol, cyclo(Leu-Ala), apicidin, and beauvericin.

In addition, lipophilic compounds may be selected from QS-21, MPLA, tentoxin, enniatin A, thiamine, ancitabine, trapoxin A, enniatin Al, cycloartenol, cypermethrin sugar, amino acids, organic acids, organic alcohol compounds and their derivatives, glycerol, organic acids having 3 or more carbon atoms and their derivatives, such as malonic acid, malic acid, oxalic acid, lactic acid, fumaric acid, tartaric acid, citric acid, quinic acid, diisopropyl azodicarboxylate, acetic acid, trioctylamine, benzoic acid, 2-phenyl-pentanoic acid, oleic acid, palmitic acid (stearic acid), sorbic acid, hexanoic acid, methyl isovalerate, heptanoyl chloride, dodecenoic acid, lauric acid, sebacic acid, pyridoxine hydrochloride and sodium undecylenate, and fatty acids.

In the present invention, the amine-based compound may be selected from alkyldiamine-based compounds including ethylenediamine, and oligomers and polymers including polyamine.

For example, among diamine-based compounds, a monomeric compound may be selected from among hydrazine, ethylenediamine, 1,3-diaminopropane, 3,5-diamino-1,2,4-triazole, cadaverine, hexamethylenediamine, bis(hexamethylene)triamine, triethylenetetramine, N,N′-bis(2-aminoethyl)-1,3-propanediamine, 2,2-bis(aminoethoxy)propane, benzidine, spermine, minoxidil, 2,2′-(ethylenedioxy)bis(ethylamine), arginine, lysine, cystamine, and cysteamine.

Among diamine-based compounds, a polymeric compound may be selected from diamine and polyamine-based polymer compound derivatives including biocompatible polymers, such as poly(ethylene glycol) bis(amine), 0,0′-bis(2-aminoethyl)octadecaethylene glycol, chitosan, PEI, collagen and the like.

The poly-gamma-glutamic acid used in the present invention may have a molecular weight of 1-15,000 kDa.

In an example of the present invention, a complex of a poly-gamma-glutamic acid having carboxyl groups with a lipophilic compound such as cholesterol was substituted with a positively charged amine group capable of reacting with a negatively charged cell surface, thereby preparing biocompatible polymer nanomicelles which can be loaded with a drug or an antigen.

As used herein, the term “poly-gamma-glutamic acid (γ-PGA) nanomicelles” refers to polymer micelles obtained by dispersing in an aqueous solution a poly-gamma-glutamic acid conjugate wherein both a lipophilic compound such as cholesterol and a positively charged compound such as a compound having an amine group are attached to poly-gamma-glutamic acid.

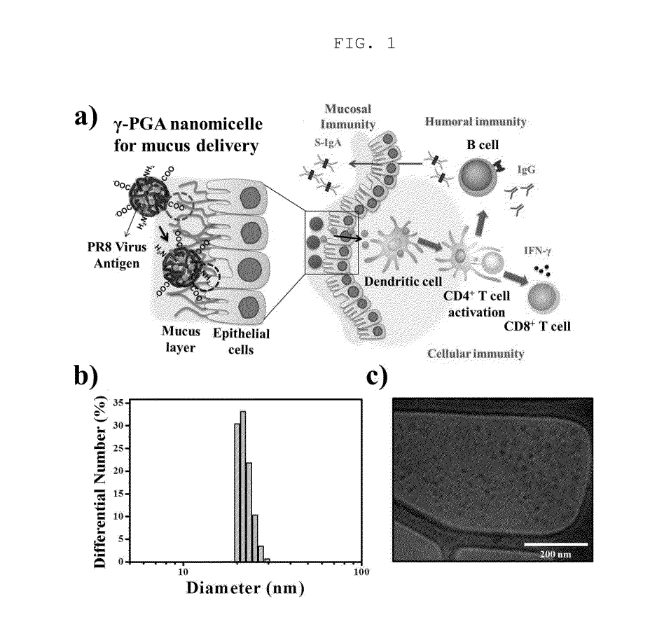

First, the characteristics of the poly-gamma-glutamic acid-cholesterol nanomicelles prepared in an example of the present invention were analyzed. When the particle diameter of the nanomicelles was measured by DLS, it was found that the nanomicelles have a diameter of 22.1±2.0 nm. Inspection of Cryo-TEM indicated that the nanomicelles are spherical poly-gamma-glutamic acid-cholesterol nanomicelles (FIG. 1). In addition, the results of NMR analysis indicated that the nanomicelles have a content of 1.7 mol % of cholesterol, and the results of elementary analysis indicated that the nanomicelles were substituted with about 28.1 mol % of an amine group. In addition, the results of dynamic light scattering analysis indicated that the nanomicelles had a surface charge of about 36.43 mV.

In another aspect, the present invention is directed to a nanomicelle drug delivery system wherein a drug selected from the group consisting of proteins, genes, peptides, compounds, antigens and natural materials is loaded in nanomicelles composed of a complex of a lipophilic compound and a poly-gamma-glutamic acid wherein a portion of carboxyl groups are substituted with an amine group.

In the present invention, the antigen may be selected from polysaccharides, live attenuated intact microorganisms, inactivated microorganisms, recombinant peptides and proteins, glycoproteins, glycolipids, lipopeptides, synthetic peptides, disrupted microorganisms, etc., but is not limited thereto.

The drug contained in the drug delivery system of the present invention can be delivered through a mucous membrane using the mucous membrane adhesive property of the drug delivery system. The mucus may be oral cavity mucosa, nasal cavity mucosa, respiratory system mucosa, eye mucosa, reproductive system mucosa, skin ulcer site or the like.

In an example of the present invention, in order to demonstrate the mucous membrane adhesive property and antigen delivery property of the poly-gamma-glutamic acid-cholesterol nanomicelles, an ¹²³I—or FITC-labeled OVA model antigen was injected intranasally to mice, and then the in vivo behavior of the OVA was analyzed by SPECT/CT imaging and immunohistofluorescence imaging techniques. SPECT/CT images showed that when OVA plus poly-gamma-glutamic acid-cholesterol nanomicelles were injected, the OVA remained in the nasal cavity even after 12 hours, but when OVA alone was injected, the OVA disappeared within 6 hours.

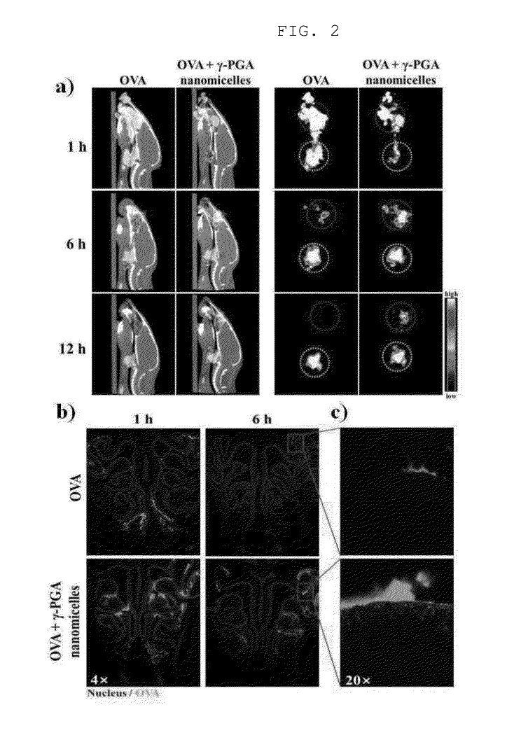

Immunohistofluorescence images indicated that when OVA plus poly-gamma-glutamic acid-cholesterol nanomicelles were injected, the OVA remained in the nasal cavity even after 6 hours, but when OVA alone was injected, the OVA disappeared after 1 hour. In addition, it was observed that when OVA plus poly-gamma-glutamic acid-cholesterol nanomicelles were injected, the OVA was effectively delivered into nasal mucous tissue, unlike when OVA alone was injected (FIG. 2).

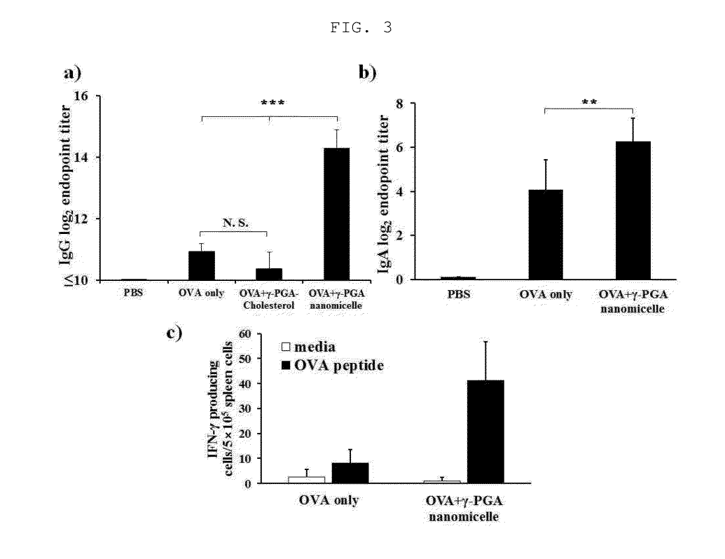

Based on the above results, the present inventors have found that the poly-gamma-glutamic acid-cholesterol nanomicelles of the present invention have the properties of adhering to nasal mucous membranes and delivering antigens. In the present invention, poly-gamma-glutamic acid-cholesterol nanomicelles loaded with the model antigen OVA were injected intranasally to mouse models, and then antigen-specific immunity in the mouse models was measured. Specifically, in order to confirm antigen-specific humoral immune responses, the production of an OVA-specific IgG antibody in mouse plasma was analyzed. As a result, it was found that the production of an IgG antibody in the mice injected with OVA plus poly-gamma-glutamic acid-cholesterol nanomicelles was 10.2 times higher than that in the control group. In addition, the production of the antibody IgA that is produced specifically in mucosal immunity was 4.5 times higher in the group administered with OVA plus poly-gamma-glutamic acid-cholesterol nanomicelles than in the control group (FIG. 3).

For reference, in the case in which an antigen was injected together with a poly-gamma-glutamic acid (γ-PGA)-cholesterol complex (whose surface is composed of carboxyl groups) wherein poly-gamma-glutamic acid is substituted only with a lipophilic group such as cholesterol, antigen-specific immune responses in this case did not greatly differ from those in the case in which an antigen alone was injected, suggesting that the delivery of the antigen through mucous membranes is inefficient (FIG. 3a). Such results indicate that the function of a positive charge derived from an amine group (NH₂) in the poly-gamma-glutamic acid (γ-PGA)-cholesterol nanomicelles is very closely associated with the mucous membrane adhesive property and drug delivery function of the nanomicelles.

Cell-mediated immune responses are known to play an important role in controlling viral infection, alleviating symptoms of diseases, and promoting recovery from diseases. Such antiviral cell-mediated immune responses are induced by introduction of cytotoxic T lymphocytes and type 1 CD4+T-helper lymphocytes (Th1) that induce immune-stimulating cytokines such as IFN-γ and IL-2.

Thus, whether the poly-gamma-glutamic acid-cholesterol nanomicelles of the present invention induce OVA antigen-specific cell-mediated immunity was examined by an IFN-γ ELISpot assay. When the number of IFN-γ-producing cells among the spleen cells of mice injected with a vaccine was measured, it was observed that the number of IFN-γ-producing cells in the group injected with the nanomicelles of the present invention was 5.2 times larger than that in the control group, suggesting that the nanomicelles of the present invention have the ability to induce cell-mediated immunity (FIG. 3).

Based on the above results, the present inventors have found that the poly-gamma-glutamic acid-cholesterol nanomicelles of the present invention are effective in inducing humoral and cell-mediated immunity. Thus, the effect of the poly-gamma-glutamic acid-cholesterol nanomicelles of the present invention was verified by using an influenza virus (influenza A/PuertoRico/8/34; PR8; H1N1) antigen.

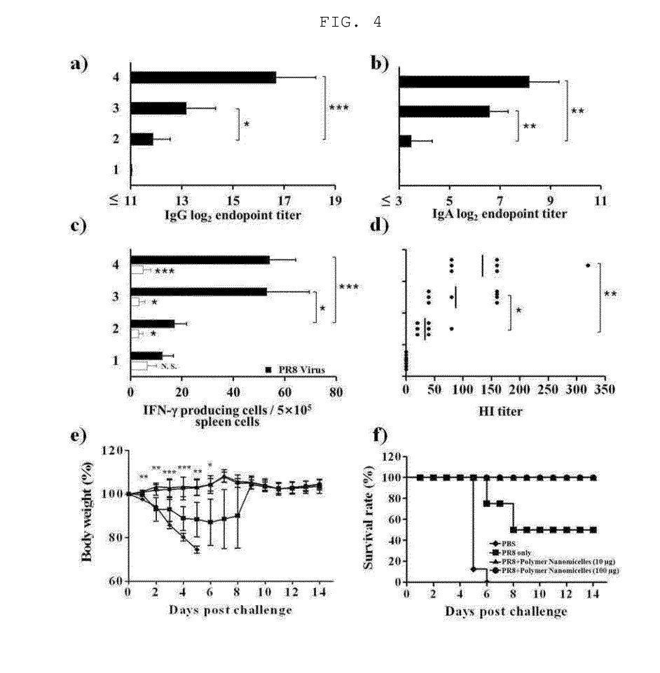

Specifically, in order to confirm the effect of inducing antigen-specific humoral immune responses, the production of PR8 antigen-specific antibodies was analyzed. As a result, it was shown that the production of IgG antibody in mice injected with PR8 antigen plus poly-gamma-glutamic acid-cholesterol nanomicelles was 28.6 times higher than that in the control group, and the production of IgA antibody was 27.6 times higher than that in the control group (FIG. 4). In addition, whether the poly-gamma-glutamic acid-cholesterol nanomicelles of the present invention induce PR8 antigen-specific cell-mediated immunity was examined by an IFN-γ ELISpot assay. Specifically, when the number of IFN-γ-producing cells among the spleen cells of mice injected with a vaccine was measured, it was found that the number of IFN-γ-producing cells in the mice was 3.2 times larger than that in the control group, suggesting that the nanomicelles of the present invention have the ability to induce cell-mediated immunity. In addition, based on the fact that antiviral antibodies interfere with the binding of the influenza virus surface protein HA to erythrocytes, the production of PR8 antigen-specific antibodies was analyzed, and as a result, it was shown that the production of the antibodies was 4 times higher than that in the control group (FIG. 4).

Based on the above results, the present inventors analyzed survival rate against viral infection. Specifically, when survival rate was analyzed using a 10-fold concentration of a virus having a lethality of 50%, a test group injected with PBS showed a lethality of 100%, and a test group injected with the PR8 antigen alone showed a lethality of 50%. However, a test group injected with PR8 antigen plus poly-gamma-glutamic acid-cholesterol nanomicelles showed a survival rate of 100% (FIG. 4).

Based on the above-described results, the present inventors have found that the poly-gamma-glutamic acid-cholesterol nanomicelles of the present invention are effective in inducing humoral and cell-mediated immunity and can increase resistance against viral infection.

### EXAMPLES

Hereinafter, the present invention will be described in further detail with reference to examples. It will be obvious to a person having ordinary skill in the art that these examples are illustrative purposes only and are not to be construed to limit the scope of the present invention.

### Example 1

### Preparation of Poly-Gamma-Glutamic Acid/Cholesterol Nanomicelles

### 1-1: Synthesis of Cholesterol-Amine

250 mmol of ethylenediamine (Sigma-Aldrich, USA) was dissolved in 250 ml of toluene. Herein, the solution was maintained at a low temperature using ice. 2.25 g of cholesterol was dissolved in 50 ml of toluene and allowed to stand for 10 minutes. Next, the cholesterol solution was added dropwise to the above-prepared ethylenediamine solution, and then immediately, the mixture solution was allowed to react with stirring at room temperature for 16 hours. After completion of the reaction, the reaction solution was washed several times with deionized water. The resulting clear organic layer was dried by using magnesium sulfate, and toluene was removed from the dried solution by rotary evaporation. The sample remaining after evaporation was rinsed several times with a mixture of 20 ml of dichloromethane and 20 ml of methanol, and filtered through a 1-μm PTFE filter. The filtered clear solution was subjected to rotary evaporation to obtain a cholesterol-amine sample as a white solid. The obtained cholesterol-amine sample was analyzed by NMR to measure the degree of bonding between cholesterol and amine. The results of the NMR analysis indicated that about 98 mole % of amine was bonded.

### 1-2: Synthesis of Poly-Gamma-Glutamic Acid-Cholesterol

1 g of poly-gamma-glutamic acid (50 KDa, Bioleaders, Korea) was dissolved in 10 ml of DMSO at 40° C. for about one day. 1 g of the cholesterol-amine prepared in Example 1-1 was dissolved in 10 ml of tetrahydrofuran (THF; Sigma-Aldrich, USA) at room temperature. The cholesterol-amine solution was slowly added dropwise to the poly-gamma-glutamic acid solution, and 1 g of carbodiimide (CDI) was added to the mixture solution which was then allowed to react at 40° C. for about one day. The reaction solution was cooled to room temperature, and then subjected to rotary evaporation to remove THF.

The solution remaining after the removal of THF was precipitated in acetone, and the solvent and the solute were sufficiently separated from each other by centrifugation to thereby remove acetone, and the remaining material was dried at 40° C. The dried sample was added to deionized water, and sodium hydrogen carbonate was added to the sample in the same molar amount as the poly-gamma-glutamic-acid used in the reaction to thereby neutralize the poly-gamma-glutamic-acid. The resulting solution was stirred with 5 g of amberlite (Sigma-Aldrich, USA) for about 2 hours to remove unreacted cholesterol and impurities. The stirred solution was filtered through a mesh to remove amberlite and was dialyzed using a cellulose membrane tube (MWCO 12,000, Sigma-Aldrich, USA) for 2 days. The dialyzed solution was freeze-dried to obtain poly-gamma-glutamic acid-cholesterol nanomicelles. The nanomicelles were analyzed by NMR to measure the amount of cholesterol introduced. The results of the NMR analysis indicated that 1.7 mol % of cholesterol was bonded.

### 1-3: Synthesis of Poly-Gamma-Glutamic Acid-Cholesterol Micelles

100 mg of the poly-gamma-glutamic acid-cholesterol complex and 0.518 ml of ethylene diamine were dissolved in 50 ml of DMSO containing 60 mg of carbonyldiimidazole dissolved therein, followed by stirring for about 24 hours. Elemental analysis was performed to quantify the amounts of C, N and H, thereby quantifying the amount of amine group introduced. In the results of the analysis, the poly-gamma-glutamic acid (γ-PGA)-cholesterol complex showed values of C (35.16±0.08%), H (4.87±0.19%) and N (7.40±0.04%), and the poly-gamma-glutamic acid-cholesterol nanomicelles (i.e., aminated γ-PGA-cholesterol) showed values of C (42.41±0.38%), H (6.95±0.13%) and N (15.90±0.14%). As a result, it could be seen that the poly-gamma-glutamic acid-cholesterol nanomicelles were substituted with about 28.1 mol % of an amine group.

### Example 2

### Characterization of Poly-Gamma-Glutamic Acid-Cholesterol Nanomicelles

The poly-gamma-glutamic acid-cholesterol nanomicelles were dispersed in distilled water at a concentration of 1 mg/mL and measured by DLS (dynamic light scattering, Otsuka, Japan). As a result, as shown in FIG. 1b), the nanomicelles had a diameter of 22.1±2.0 nm. In addition, the poly-gamma-glutamic acid-cholesterol nanomicelles were analyzed by Cryo-TEM, and as a result, as shown in FIG. 1c), the nanomicelles were spherical poly-gamma-glutamic acid-cholesterol nanomicelles. Furthermore, the results of NMR analysis of the poly-gamma-glutamic acid-cholesterol nanomicelles indicated that the nanomicelles had a content of 1.7 mol % of cholesterol, and the results of elemental analysis of the poly-gamma-glutamic acid-cholesterol nanomicelles indicated that the nanomicelles were substituted with about 28.1 mol % of an amine group. In addition, the results of measurement of the poly-gamma-glutamic acid-cholesterol nanomicelles by dynamic light scattering indicated that the nanomicelles had a surface charge of about 36.43 mV.

### Example 3

### Loading of OVA into Poly-Gamma-Glutamic Acid-Cholesterol Nanomicelles

In order to load OVA (ovalbumin, Sigma-Aldrich, USA) into the poly-gamma-glutamic acid-cholesterol nanomicelles prepared in Example 1, 8 mg/mL of the nanomicelles were mixed with OVA at a mass ratio of 5:1, and the mixture was allowed to react for 1 hour, thereby obtaining poly-gamma-glutamic acid-cholesterol nanomicelles loaded with OVA. In order to find the ratio at which OVA was completely loaded without free OVA, 8 mg/mL of the poly-gamma-glutamic acid-cholesterol nanomicelles and 10 mg/mL of OVA were reacted at a mass ratio ranging from 2:1 to 9:1, and the reaction products were analyzed on polyacrylamide gel (SDS free gel). As a result, it was found that OVA was completely loaded at a ratio of 5:1.

### Example 4

### Preparation of Poly-Gamma-Glutamic Acid-Cholesterol Nanomicelles Loaded with Iodine (I)

### 4-1: Preparation of Poly-Gamma-Glutamic Acid-Cholesterol Nanomicelles Loaded with Tyrosine

Because iodine has a high binding affinity for the phenol ring of tyrosine, iodine can be introduced into nanomicelle structures by bonding tyrosine into nanomicelle structures. Specifically, the poly-gamma-glutamic acid-cholesterol nanomicelles prepared in Example 1 were dissolved in water, and then EDC (1-ethyl-3-[3-dimethylaminopropyl] carbodiimide hydrochloride, Sigma-Aldrich, USA), NHS (N-hydroxysuccinimide, Sigma-Aldrich, USA) and tyrosine amide (Sigma-Aldrich, USA) were added thereto, thereby forming an amide bond between the carboxyl group of poly-gamma-glutamic acid and the amine group of tyrosine amide. The mixture was dialyzed and freeze-dried to remove unreacted materials, thereby obtaining a nanogel sample as powder. The results of NMR analysis of tyrosine attached to the nanomicelles indicated that about 0.7 mole % of tyrosine was introduced into the nanomicelles.

### 4-2: Preparation of Nanomicelles Loaded with Iodine

The tyrosine-loaded nanomicelles were dissolved in PBS and mixed with an isotope ion solution (Na¹²³I, Pierce, USA), and the mixture was allowed to react with stirring in an iodination tube at room temperature for about 1 hour.

The reaction solution was loaded onto a PD10 column (Sigma-Aldrich, USA), and the eluate was fractioned into a volume of 1 ml or 0.5 ml. The ¹²³I-activity of each of the eluate fractions was measured by a gamma-counter, and among the eluate fractions, an eluate fraction showing the highest activity was selected and used.

### 4-3: Measurement of Mucous Adhesive Property (SPECT/CT) of Poly-Gamma-Glutamic Acid-Cholesterol Nanomicelles in Mouse Models

After the reaction of the tyrosine-loaded nanomicelles with iodine, an eluate fraction showing the highest activity was selected. The selected eluate fraction was diluted with PBS to a final concentration of 500 μg/ml in view of ¹²³I-activity to be injected per mouse.

The amount of sample injected for SPECT/CT imaging was such that the sample would have a ¹²³I-activity of about 200 μCi or more in 40 μl upon nasal administration. 40 μl of a sample was injected into each mouse using a tip for nasal administration, and at 1, 6 and 12 hours after injection, SPECT/CT imaging was started. SPECT/CT imaging was performed for about 1 hour, in which CT imaging was performed for 10 minutes, followed by SPECT imaging for 50 minutes. Because the amount of sample injected intranasally is limited to 40 μl or less, the concentration of the sample is necessarily required to ensure ¹²³I-activity for imaging. ¹²³I-labeled nanomicelles were centrifuged, filtered (Ultracel, 10 kDa) and concentrated, thereby ensuring activity required for imaging per 40 μl. The sample was injected into both nasal cavities alternately at intervals of a few minutes depending on the size and condition of the mouse.

As a result, as shown in FIG. 2, the SPECT/CT images showed that when OVA plus poly-gamma-glutamic acid-cholesterol nanomicelles were injected, OVA remained in the nasal cavity even after 12 hours, but when OVA alone was injected, OVA disappeared within 6 hours.

Immunohistofluorescence images showed that when OVA plus poly-gamma-glutamic acid-cholesterol nanomicelles were injected, OVA remained in the nasal cavity even after 6 hours, but OVA alone was injected, OVA disappeared after 1 hour. In addition, it was observed that when OVA plus poly-gamma-glutamic acid-cholesterol nanomicelles were injected, the OVA was effectively delivered into nasal mucous tissue, unlike when OVA alone was injected.

### Example 5

### Animal Immunization Test

### 5-1: Animals and Animal Immunization

An animal immunization test was performed in order to examine the in vivo immune characteristics of the antigen-loaded poly-gamma-glutamic acid-cholesterol nanomicelles prepared in Example 3. In the animal test, specific pathogen-free 6-week-old female C57BL/6 mice (Coretech, Korea) were used, and all the experimental protocols were performed with the approval of the Laboratory Animal Center, Chungnam National University. Specifically, 2.5% avertin (2,2,2-tribromoethanol-tert-amyl alcohol, Sigma-Aldrich, USA) solution was injected intraabdominally to mice in an amount of 0.01 ml/g of body weight to anesthetize the mice, and 20 μl of antigen-loaded poly-gamma-glutamic acid-cholesterol nanomicelles were injected alternately into both nasal cavities of the mice (10 μl for each nasal cavity). For a control group, an antigen alone contained in PBS was injected intranasally. Immunization of the mice was performed three times at 7-day intervals between injections.

### 5-2: Sampling and Antibody ELISA Assay for Antibody Measurement

At 1 week after immunization of the animals treated in Example 5-1, blood was collected from eye blood vessels, and the collected blood was allowed to stand at room temperature for about 1 hour. Next, the blood was centrifuged at 4° C., and the supernatant was collected. Using this solution, the production of antigen-specific IgG was measured. To collect nasal mucus, at 1 week after the third immunization, the mice were sacrificed by cervical dislocation, and then 200 μl of PBS was injected into the nasal cavity and recovered again. Using this solution, the production of antigen-specific IgA was measured.

The production of antigen-specific IgG and IgA was measured by an ELISA assay. The ELISA assay of the mouse antibodies was performed according to the manufacturer's instructions. First, antigen protein was diluted in 0.05M carbonate-bicarbonate buffer (pH 9.6) at a concentration of 1 μg/ml, and 100 μl of the dilution was coated on each well of an ELISA plate. The plate was incubated overnight at 4° C., washed three times with PBS-T (Biorad, USA), and then blocked with 2% BSA. This procedure was performed at 37° C. for 1 hour. 100 μl of each of plasma, diluted with PBS after washing, and nasal mucus serially diluted 8-fold, was added to each well and incubated at 37° C. for 2 hours. Thereafter, the plate was incubated with HRP-conjugated secondary antibody for 1 hour, and then TMB reagent (Biorad, USA) was added thereto to induce color development. After 30 minutes, 2N sulfuric acid was added to the plate to stop the reaction, and then the absorbance at a wavelength of 450 nm was measured by using an ELISA reader. The titer of the antibody was determined at an OD value of 0.1.

As a result, as shown in FIG. 3a, the production of IgG antibody in the mouse group injected with OVA plus poly-gamma-glutamic acid-cholesterol nanomicelles was 10.2 times higher than that in the control group, and in the case of the poly-gamma-glutamic acid-cholesterol complex whose surface was not substituted with an amine group, an increase in the production of the antibody could not be observed.

As shown in FIG. 3b, the production of the antibody IgA that is produced specifically in mucosal immunity was 4.5 times higher in the mouse group injected with OVA plus poly-gamma-glutamic acid-cholesterol nanomicelles than in the control group.

### 5-3: ELISpot Assay

At 1 week after immunization of the animals treated in Example 5-1, the mice were sacrificed by cervical dislocation. Five mice were selected from each mouse group, and the spleen was removed from each mouse. The spleen tissue was transferred to a sterilized Petri dish and mashed through a cell strainer to isolate cells from the tissue capsule. A suspension of single spleen cells was prepared. A mouse IFN-γ ELISpot (Nunc, Netherands) assay was performed according to the manufacturer's instructions. Specifically, IFN-γ antibody (5 μg/ml) was added to an ELISpot plate, and the plate was incubated overnight, and then blocked with complete medium (RPMI supplemented with 10% fetal bovine serum) for 2 hours. The spleen cells (5×10⁵ cells) were stimulated in complete medium at a concentration of 10 μl/ml per peptide in a total volume of 200 μl at 37° C. and 5% CO₂ for 60 hours. Next, the cells were washed and treated with HRP-conjugated secondary antibody for 2 hours, and then incubated with AEC (3-amino-9-ethyl-carbozole, Sigma-Aldrich, USA) for 15 minutes to induce color development. Next, spots were counted with a CTL-immune spot reader unit (Molecular Devices, USA). The results are expressed as the mean±SD of spot forming cells (SFC) per million input spleen cells.

As a result, as shown in FIG. 3c, the number of IFN-γ-producing cells in the spleen cells of the mice injected with the vaccine was 5.2 times higher than that of the control group, indicating that the poly-gamma-glutamic acid-cholesterol nanomicelles of the present invention have the ability to induce cell-mediated immunity.

### 5-4: Hemagglutination Assay

Measurement of antibody titer in the mouse serum of each group was performed by a haemagglutination inhibition (HI) assay for virus. Each serum was treated with RDE (receptor-destroying enzyme, Denka Seiken, Japan), extracted from Vibrio cholerae, at a volume ratio of 1:10, followed by incubation at 37° C. for 16 hours. 25 μl of a sample obtained by removing the activity of nonspecific receptors from the serum was serially diluted two-fold in a 96-well round-bottom plate. Next, the same volume of 4 HAU virus was added to the serum sample and incubated in an incubator at 37° C. for 30 minutes. Finally, 50 μl of PBS containing 0.5% chick erythrocytes (tRNA) was added to each well, followed by incubation at room temperature for 40 minutes. The antibody titer was calculated in 50 μl of the diluted serum and expressed as the N value in log 10N=10N (FIG. 4d).

### Example 6

### Assay of Survival Rate Against Viral Infection

In this Example, the lethality of test animals infected with influenza virus was assayed in order to examine the effect of PR8 virus antigen-loaded poly-gamma-glutamic acid-cholesterol nanomicelles on the enhancement of immunity against avian influenza virus.

### 6-1: Preparation of Virus

Influenza virus used as a pathogen was an H1N1 influenza virus strain (A/Puerto Rico/8/34(H1N1), Korea Research Institute of Bioscience and Biotechnology (KRIBB), Korea) showing high pathogenicity in mice. The virus strain was amplified in the fertilized egg of 10-11-day-old white leghorn eggs, and then used in the experiment. As test animals, 6-week-old female C57BL/6 mice (Koatech, Korea) were used. The purification of the viral strain was performed in the following manner. First, the isolated virus was diluted in antibiotic-containing PBS and inoculated into the fertilized egg of a 10-day-old white leghorn chicken. Then, the inoculated virus was stationary-incubated at 37° C. for 48 hours, after which the allantoic fluid of the fertilized egg was taken to obtain amplified virus.

### 6-2: Animal Immunization and Viral Infection

As a control group, mice injected intranasally with influenza virus alone, and mice injected intranasally with PR8 virus antigen (M.-S. Lee, A. Hu, Trends Microbiol. 20: 103, 2012) were used. In the case of a test group, PR8 virus antigen-loaded poly-gamma-glutamic acid-cholesterol nanomicelles were administered intranasally, and on the next day, influenza virus was administered. For viral infection, each test animal was anesthetized by intraabdominal administration of 200 μl of avertin, and then 30 μl of virus was administered intranasally to each mouse at a dose 10 times that of virus having a lethality of 50% (10×LD₅₀). Up to 2 weeks after viral infection, the body weight of the mice was measured, and the lethality of the mice was assayed.

As a result, it was shown that the production of IgG antibody in the mice injected with PR8 antigen plus poly-gamma-glutamic acid-cholesterol nanomicelles was 28.6 times higher than that in the control, and the production of IgA antibody in the mice was 27.6 times higher than in the control group (FIGS. 4a and 4b). In addition, whether the poly-gamma-glutamic acid-cholesterol nanomicelles induce PR8 antigen-specific cell-mediated antigen immunity was examined by an IFN-γ ELISpot assay. When the number of IFN-γ-producing cells in the spleen cells of the mice injected with the vaccine was measured, it was shown that the number of IFN-γ-producing cells in the mice was 3.2 times higher than that in the control group, indicating that the poly-gamma-glutamic acid-cholesterol nanomicelles have the ability to induce cell-mediated immunity (FIG. 4c). In addition, based on the fact that antiviral antibodies interfere with the binding of the influenza virus surface protein HA to erythrocytes, when the production of PR8 antigen-specific antibodies in the mice injected with PR8 antigen plus poly-gamma-glutamic acid-cholesterol nanomicelles was analyzed, it was shown that the production of the antibodies in the mice was 4 times higher than that in the control group (FIG. 4d).

Based on the above results, survival rate was analyzed using a 10-fold concentration of virus having a lethality of 50%. As a result, the test group injected with PBS showed a lethality of 100%, and the test group injected with only the PR8 antigen showed a lethality of 50%. However, the test group injected with PR antigen plus poly-gamma-glutamic acid-cholesterol nanomicelles showed a survival rate of 100% (FIG. 4f).

### INDUSTRIAL APPLICABILITY

As described above, according to the present invention, the nanomicelle drug delivery system based on poly-gamma-glutamic acid that is a natural biopolymer can be used for the delivery of a drug to mucous membranes to thereby increase the in vivo stability and effectiveness of the drug.

Although the present invention has been described in detail with reference to the specific features, it will be apparent to those skilled in the art that this description is only for a preferred embodiment and does not limit the scope of the present invention. Thus, the substantial scope of the present invention will be defined by the appended claims and equivalents thereof.

1. Nanomicelles composed of a complex of a lipophilic compound and a poly-gamma-glutamic acid wherein a portion of carboxyl groups are substituted with an amine group.

2. The nanomicelles of claim 1, wherein the lipophilic compound is selected from the group consisting of cholesterols and their derivatives, aliphatic compounds having 3 to 21 carbon atoms (C₃-C₂₁), and aromatic compounds containing 1 to 10 benzene groups.

3. The nanomicelles of claim 1, wherein the poly-gamma-glutamic acid has a molecular weight of 1-15,000 kDa.

4. A method for preparing nanomicelles composed of a complex of a lipophilic compound and a poly-gamma-glutamic acid wherein a portion of carboxyl groups are substituted with an amine group, the method comprising the steps of:
(a) preparing a lipophilic compound-amine complex;
(b) mixing a poly-glutamic acid solution with a lipophilic compound-amine complex solution to prepare a poly-glutamic acid-lipophilic compound complex; and
(c) treating the poly-glutamic acid-lipophilic compound complex with an amine-based compound to substitute the carboxyl group of the poly-gamma-glutamic acid with an amine group, thereby preparing nanomicelles composed of a complex of a lipophilic compound and a poly-gamma-glutamic acid wherein a portion of carboxyl groups are substituted with an amine group.

5. The method of claim 4, wherein the lipophilic compound is selected from the group consisting of cholesterols and their derivatives, aliphatic compounds having 3 to 21 carbon atoms (C₃-C₂₁), and aromatic compounds containing 1 to 10 benzene groups.

6. The method of claim 4, wherein the poly-gamma-glutamic acid has a molecular weight of 1-15,000 kDa.

7. The method of claim 4, wherein the amine-based compound is selected from alkyldiamine-based compounds including ethylenediamine, and oligomers and polymers including polyamine.

8. A nanomicelle drug delivery system wherein a drug selected from the group consisting of proteins, genes, peptides, compounds, antigens and natural materials is loaded in the nanomicelles of claim 1.

9. The nanomicelle drug delivery system of claim 8, wherein the antigen is selected from the group consisting of polysaccharides, live attenuated intact microorganisms, inactivated microorganisms, recombinant peptides and proteins, glycoproteins, glycolipids, lipopeptides, synthetic peptides, and disrupted microorganisms.

10. The nanomicelle drug delivery system of claim 8, wherein the drug is delivered through a mucous membrane.

11. The nanomicelle drug delivery system of claim 8, wherein the mucous membrane is selected from the group consisting of oral cavity mucosa, nasal cavity mucosa, respiratory system mucosa, eye mucosa, reproductive system mucosa, and skin ulcer site.
